(19) United States
(12) Patent Application Publication
(10) Pub. No.: US 2009/0118253 A1
(43) Pub. Date: May 7, 2009

(54) COMPOSITIONS AND METHODS FOR TREATING DYSFUNCTIONAL UTERINE BLEEDING

(75) Inventors: Andre van As, West Chester, PA (US); Joseph S. Podolski, The Woodlands, TX (US); Ronald D. Wiehle, Houston, TX (US)

Correspondence Address: HOWREY LLP - East, C/O IP DOCKETING DEPARTMENT, 2941 FAIRVIEW PARK DR, SUITE 200, FALLS CHURCH, VA, 22042-2924, US

(73) Assignee: REPROS THERAPEUTICS INC., The Woodlands, TX (US)

(21) Appl. No.: 12/245,089

(22) Filed: Oct. 3, 2008

Related U.S. Application Data
(60) Provisional application No. 60/985,528, filed on Nov. 5, 2007.

(51) Int. Cl. A61K 31/573

(52) U.S. Cl. 514/179

(57) ABSTRACT

The subject matter of the instant invention is pertinent to the field of hormone therapy. More specifically, the subject matter of the instant invention concerns methods of treating dysfunctional uterine bleeding. The instant invention is also relevant to the treatment and/or prevention of anemia in patients with dysfunctional uterine bleeding. Compositions for practicing the methods, comprising progesterone antagonists are also disclosed. Embodiments of the instant invention also disclose methods for identifying new selective progesterone receptor modulators for practicing disclosed methods of treatment.

### CROSS REFERENCE TO RELATED APPLICATIONS

The present application claims the benefit of U.S. Provisional Patent Application No. 60/985,528, filed Nov. 5, 2007, which is incorporated herein by reference.

### FIELD OF THE INVENTION

The present invention relates to compositions and methods for treating dysfunctional uterine bleeding. More specifically, the present invention relates to compositions comprising one or more progesterone antagonists for treating and/or preventing anemia in patients with dysfunctional uterine bleeding.

### BACKGROUND OF THE INVENTION

Dysfunctional uterine bleeding (DUB) is the most common cause of abnormal vaginal bleeding during a woman's reproductive years. The incidence of dysfunctional uterine bleeding is high and represents one of the most frequent reasons for gynecological consultation for women of reproductive age. The diagnosis of DUB is made only after other organic and structural causes of abnormal uterine bleeding are ruled out. During a normal menstrual cycle, menstruation occurs for about 2-7 days with an average blood loss of about 35-150 ml.

Approximately 90% of DUB results from anovulation and 10% occur with ovulatory cycles. During an anovulatory cycle, the corpus luteum fails to form, which causes failure of normal progesterone secretion. Estradiol is then unopposed, stimulating overgrowth of the endometrium which eventually outgrows its blood supply leading to necrosis and ultimately overproduction of uterine blood flow.

In ovulatory DUB, prolonged progesterone secretion causes irregular shedding of the endometrium, presumably related to a constant low level of estrogen that is around the bleeding threshold. This causes spotting as portions of the endometrium degenerate.

Dysfunctional uterine bleeding may take the form of menorrhagia, metrorrhagia, menometrorrhagia, spotting, or polymenorrhea, oligomenorrhea, or amenorrhea. Menorrhagia is defined as prolonged (over 7 days) or excessive (over 80 ml daily) uterine bleeding occurring at regular intervals. Metrorrhagia is defined as uterine bleeding occurring at irregular and more frequent than normal intervals. Menometrorrhagia is defined as prolonged or excessive uterine bleeding occurring at irregular and more frequent than normal intervals. Spotting is defined as uterine bleeding of variable amounts occurring between regular menstrual periods. Polymenorrhea is defined as uterine bleeding occurring at regular intervals of less than 21 days. Oligomenorrhea is defined as uterine bleeding occurring at intervals of 35 days to 6 months. Amenorrhea is defined as lack of uterine bleeding for at least 6 months.

Because dysfunctional uterine bleeding is often caused by an imbalance of one or more of the hormones related to ovulation (i.e., estrogen or progesterone), it is most common at the extremes of a woman's reproductive years, but may occur at any time during her reproductive life. DUB is most likely to be severe during adolescence or menopause, where follicle-stimulating disorders, anovulation and follicle persistence may occur in combination.

Anemia may be a potential complication for women with dysfunctional uterine bleeding. Indeed, women with menorrhagia are at significant risk of developing iron deficiency anemia and often present with serum hemoglobin level of less than 12 mg per dl.

Typical treatments for dysfunctional uterine bleeding include progesterone (e.g. 10 mg medroxyprogesterone acetate daily for 10-14 days), high doses of estrogen/progesterone combinations (for 10-14 days), nonsteroidal cyclooxygenase inhibitors and gonadotropin-releasing hormone agonists.

Each of the current treatment strategies is associated with one or more known risks and if unsuccessful, surgical procedures such as hysterectomy and uterine curettage may be required.

### SUMMARY OF THE INVENTION

The instant invention provides methods for treating dysfunctional uterine bleeding, comprising administering to a patient in need thereof an amount of a progesterone antagonist effective to treat dysfunctional uterine bleeding. The progesterone antagonist may be a pure antiprogestin or a selective progesterone receptor modulator (SPRM). In a preferred embodiment, the progesterone antagonist has low affinity for glucocorticoid receptor. In another preferred embodiment, administration of the progesterone antagonist to a female reduces luteal phase progesterone levels in the female. In yet another preferred embodiment, administration of the progesterone antagonist to a female does not substantially lower estrogen levels in the female. In yet another preferred embodiment, compositions of the invention are administered chronically to a patient with dysfunctional uterine bleeding.

In related aspects of the invention, the dysfunctional uterine bleeding is a form selected from the group consisting of: menorrhagia, metrorrhagia, menometrorrhagia, spotting, polymenorrhea, oligomenorrhea, and amenorrhea.

In yet another aspect, the instant invention provides methods of treating and/or preventing anemia in a patient with dysfunctional uterine bleeding, comprising administering to the patient a composition comprising at least one progesterone antagonist in an amount effective to prevent anemia in said patient. In one embodiment, the composition is administered chronically to a patient with dysfunctional uterine bleeding in order to treat and/or prevent anemia.

### BRIEF DESCRIPTION OF THE DRAWINGS

FIG. 1 is a graph depicting the effect of selective progesterone receptor modulators on serum cortisol in rats.

FIG. 2 is a graph depicting the dose-dependent effect of CDB-4124 on serum cortisol in rats.

### DETAILED DESCRIPTION OF THE INVENTION

The term “effective amount” means an amount of the composition's active component sufficient to achieve the desired effect which may be, e.g., treatment of dysfunctional uterine bleeding or prevention and/or treatment of anemia associated with dysfunctional uterine bleeding.

The term “selective progesterone receptor modulators” means compounds that affect functions of progesterone receptor in a tissue-specific manner. The compounds act as progesterone receptor antagonists in some tissues (for example, in the uterus) and as progesterone receptor agonists in other tissues.

The terms “treat” or “treatment” refer to both therapeutic treatment and prophylactic or preventative measures, wherein the object is to prevent or slow down (lessen) an undesired physiological change or disorder. For purposes of the present invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The term “progesterone agonist” means a compound that binds to a progesterone receptor and mimics the action of the natural hormone.

The term “progesterone antagonist” means a compound that binds to a progesterone receptor and inhibits the effect of progesterone.

The term “suppress” or “suppresses” or “suppressing” used herein in reference to proliferation of endometrial tissue means that mitotic proliferation of endometrial tissue is suppressed upon administration of a progesterone antagonist relative to untreated endometrial tissue under identical conditions and is to be distinguished from cell death via, e.g., apoptosis. The activity of a progesterone antagonist in suppressing endometrial mitotic proliferation may be tested, e.g., in a uterine cell line by, e.g., comparing the incorporation of bromodeoxyuridine (BrdU) in cells treated with a progesterone antagonist to control (untreated) cells.

The term “not substantially thickened” as used herein in reference to a female's endometrium means that the female's endometrium does not exceed 19 mm in thickness during the administration period, as measured by ultrasound. Thus, it is considered that some thickening may occur during the administration period so long as the female's endometrium does not exceed 19 mm in thickness. Preferably, the female's endometrium is less than 15 mm in thickness, more preferably is less than 10 mm in thickness during the administration period. The female's endometrium may thicken by less than 100% relative to baseline measurements, more preferably thickens by less than 50% relative to baseline measurements, and most preferably thickens by less than 25% relative to baseline measurements.

The term “not substantially reduced” as used herein in reference to hormone levels in a female means that hormone levels are maintained within the normal range during administration of compositions of the invention. Thus, it is considered that some reduction in a hormone level may occur so long as the hormone level is maintained within the normal range.

The term “not substantially increased” as used herein in reference to hormone levels in a female means that hormone levels are maintained within the normal range during administration of compositions of the invention. Thus, it is considered that some elevation in a hormone level may occur so long the hormone level is maintained within the normal range.

The present invention relates to the use of progesterone antagonists at doses effective to treat dysfunctional uterine bleeding and/or anemia associated therewith. The methods arise from the unexpected finding that certain progesterone antagonists exhibit an inverse dose dependent effect on endometrial thickening, while maintaining estrogen levels within the normal range. Specifically, it has been found that chronic administration of a high dosage of the antiprogestin/SPRM CDB-4124 suppresses endometrial thickening while lower doses fail to suppress endometrial thickening and even tend to promote endometrial thickening, despite similar levels of estrogen observed at high and low dosages. This is particularly surprising in view of the inability of the antiprogestin/SPRM RU 486 to suppress endometrial proliferation at a high dosage, demonstrated below, and several recent reports that chronic administration of high doses of antiprogestins promotes endometrial thickening, presumably due to the effects of unopposed estrogen. Because endometrial thickening is a key feature leading to blood loss upon menses, the ability of compounds of the instant invention to suppress endometrial thickening makes them unexpectedly effective in both treating dysfunctional uterine bleeding and preventing anemia in women with dysfunctional uterine bleeding.

The suppression of endometrial thickening during a treatment regimen comprising the daily administration of an effective amount of a progesterone antagonist over a six month period, described below, demonstrates the usefulness of such progesterone antagonists where chronic/long-term administration is desired. In this regard, methods of the invention may comprise administering a composition comprising an amount of a progesterone antagonist sufficient for suppressing endometrial thickening for an administration period of least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 or more days. The composition may also be administered for an administration period of least 1, 2, 3, 4, 5, 6, 7, 8, 9, 10, 11, 12 or more months. The composition may also be administered for an administration period of at least 1, 2, 3, 4, 5, 6, 7, 8, 9, 10 or more years. During the administration period, the composition may be administered daily or periodically such as every other day, every other month, and the like. The composition may also be administered intermittently. For example, the composition may be administered for an administration period of 1, 2, 3, 4, 5 or more months, followed by a period of discontinuance, followed by an administration period of 1, 2, 3, 4, 5 or more months, and so on.

By “intermittent administration” it is meant a period of administration of a therapeutically effective dose of progesterone antagonist, followed by a time period of discontinuance, which is then followed by another administration period and so forth.

By “period of discontinuance” or “discontinuance period” it is meant a discontinuing of the daily, weekly, monthly or therebetween administration of progesterone antagonist. The time period of discontinuance may be longer or shorter than the administration period but is always longer than the dosing interval during the administration period. For example, where the administration period comprises daily, weekly, or monthly dosing, the discontinuance period is at least 2 days, at least 8 days or at least 32 days, respectively. Thus, the discontinuance period maybe at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or more days.

In one embodiment, a female patient with dysfunctional uterine bleeding is administered a composition comprising a progesterone antagonist in an amount effective to treat dysfunctional uterine bleeding in the patient. In a related embodiment, the dysfunctional uterine bleeding is selected from the group consisting of: menorrhagia, metrorrhagia, menometrorrhagia, spotting, polymenorrhea, oligomenorrhea, and amenorrhea.

In another embodiment, the present invention provides methods for treating and/or preventing anemia in a patient with dysfunctional uterine bleeding using one or more progesterone antagonists. In a related embodiment, the progesterone antagonist is administered prior to, during, or subsequent to the administration of one or more iron supplements.

In a preferred embodiment of each method of the invention, administration of the progesterone antagonist to a female does not substantially reduce estrogen levels in the female. Thus the present invention provides an advantage over current therapies for the treatment of endometriosis which often employ gonadotropin-releasing hormone (GnRH) agonists such as Lupron® (leuprolide acetate).

In another preferred embodiment of each method of the invention, administration of the progesterone antagonist to a female does not substantially increase progesterone levels in the female. More preferably, administration of the progesterone antagonist to a female reduces progesterone levels in the female, particularly luteal phase progesterone levels.

In yet another preferred embodiment of each method of the invention, the progesterone antagonist exhibits reduced affinity for the glucocorticoid receptor. More preferably, the binding affinity of the progesterone antagonist for the progesterone receptor is at least 1.5 times greater than the binding affinity of the progesterone antagonist for the glucocorticoid receptor.

Any known progesterone antagonist with characteristics of the compounds described above can be used by an artisan practicing the instant invention. Particularly useful compounds include those disclosed in U.S. Pat. No. 6,900,193, hereby incorporated by reference in its entirety, as well as those disclosed in U.S. Pat. No. 6,861,415, hereby incorporated by reference in its entirety, that are 21-substituted 19-norpregnanes with a general formula:

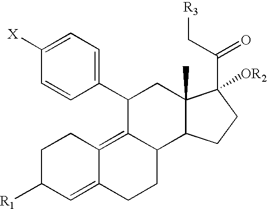

wherein:
X may be, for example alkyl, alkenyl, alkynyl, hydrogen, halo, monoalkylamino or dialkylamino, such as N,N-dimethylamino;
R₁ may be, for example O, NOH or NO-methyl;
R₂ may be, for example hydrogen or acetyl; and
R₃ may be, for example methyloxy, formyloxy, acetoxy, acyloxy, S-alkoxy, acetyltheonyl, glycimate, vinyl ether, acethyloxymethyl, methyl carbonate, halogens, methyl, hydroxy, and ethyloxy.
   The examples of 21-substituted 19-norpregnanes include, but are not limited to, the following 24 compounds disclosed below.
1. CDB-4247 (21-propio[[1]]nyloxy-17α-acetoxy-1 1β-(4 N,N-dimethylaminophenyl)-19-norpregna-4,9-diene-3,20-dione) with the following structural formula:

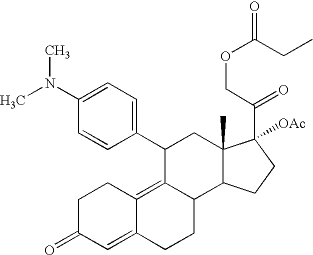

2. CDB-4361(21-vinyl ether-17α-acetoxy-11β-(4 N,N-dimethylaminophenyl)-19-norpregna-4,9-diene-3,20-dione) with the following structural formula:

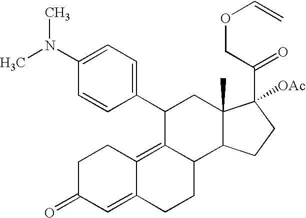

3. CDB-4059 (21-acetoxy-17α-acetoxy-11β-(4 N,N-dimethylaminophenyl)-19-norpregna-4,9-diene-3,20-dione) with the following structural formula:

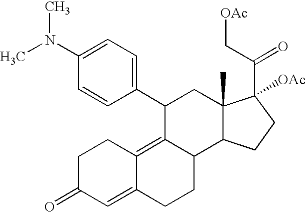

4. CDB-4124 (21-methoxy-17α-acetoxy-11β-(4 N,N-dimethylaminophenyl)-19-norpregna-4,9-diene-3,20-dione) with the following structural formula:

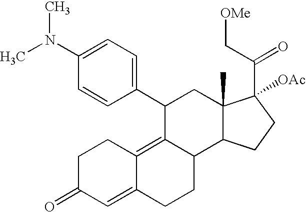

5. CDB-4031(21-bromine-17α-acetoxy-11β-(4 N,N-dimethylaminophenyl)-19-norpregna-4,9-diene-3,20-dione) with the following structural formula:

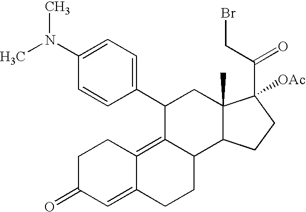

6. CDB-3876 (21-chlorine-17α-acetoxy-11β-(4 N,N-dimethylaminophenyl)-19-norpregna-4,9-diene-3,20-dione) with the following structural formula:

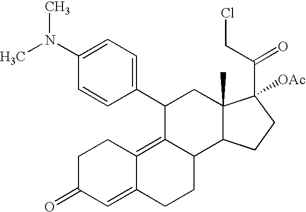

7. CDB-4058 (21-flourine-17α-acetoxy-11β-(4 N,N-dimethylaminophenyl)-19-norpregna-4,9-diene-3,20-dione) with the following structural formula:

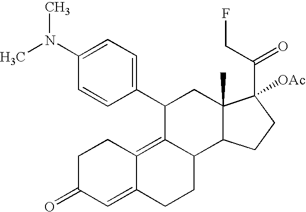

8. CDB-4030 (21-methyl-17α-acetoxy-11β-(4 N,N-dimethylaminophenyl)-19-norpregna-4,9-diene-3,20-dione) with the following structural formula:

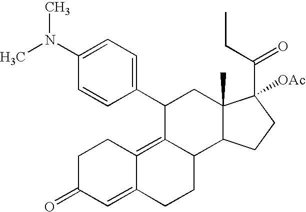

9. CDB-4152 (21-hydroxy-17α-acetoxy-11β-(4 N,N-dimethylaminophenyl)-19-norpregna-4,9-diene-3,20-dione) with the following structural formula:

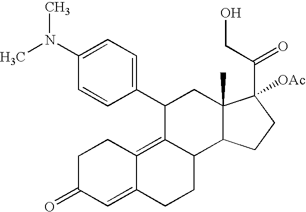

10. CDB-4167 (21-ethyloxy-17α-acetoxy-11β-(4 N,N-dimethylaminophenyl)-19-norpregna-4,9-diene-3,20-dione) with the following structural formula:

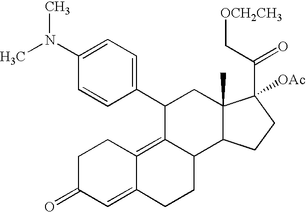

11. CDB-4101 (21-methoxythio-17α-acetoxy-11β-(4 N,N-dimethylaminophenyl)-19-norpregna-4,9-diene-3,20-dione) with the following structural formula:

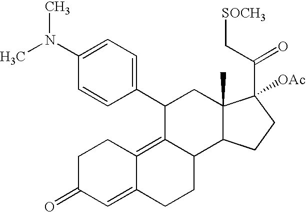

12. CDB-4110 (21-acetonide-17α-acetoxy-11β-(4 N,N-dimethylaminophenyl)-19-norpregna-4,9-diene-3,20-dione) with the following structural formula:

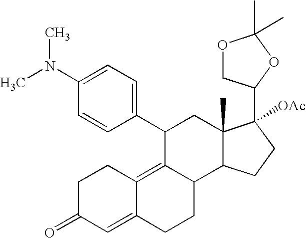

13. CDB-4111 (21-BMD-17α-acetoxy-11β-(4 N,N-dimethylaminophenyl)-19-norpregna-4,9-diene-3,20-dione) with the following structural formula:

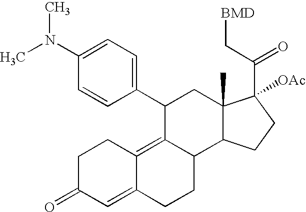

14. CDB-4125 (2 11-(Cyp*-hydroxy)-17α-acetoxy-11β-(4 N,N-dimethylaminophenyl)-19-norpregna-4,9-diene-3,20-dione) with the following structural formula:

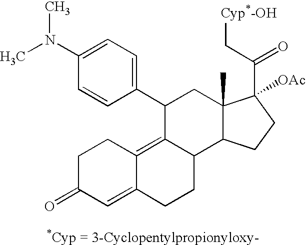

15. CDB-4205 (3-hydroxyamino-2 1-methoxy-17α-acetoxy-11β-(4 N,N-dimethylaminophenyl)-19-norpregna-4,9-diene-3,20-dione) with the following structural formula:

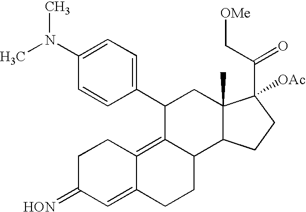

16. CDB-4206 (3-hydroxyamino-21-acetoxy-17α-acetoxy-11β-(4 N,N-dimethylaminophenyl)-19-norpregna-4,9-diene-3,20-dione) with the following structural formula:

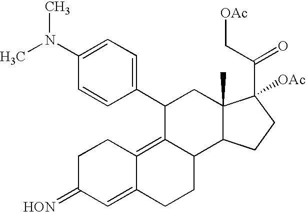

17. CDB-4226 (3-hydroxyamino-21-ethyloxy-17α-acetoxy-11β-(4 N,N-dimethylaminophenyl)-19-norpregna-4,9-diene-3,20-dione) with the following structural formula:

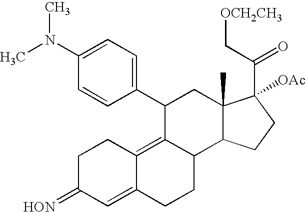

18. CDB-4262 (3-methoxyamino-21-ethyloxy-17α-acetoxy-11β-(4 N,N-dimethylaminophenyl)-19-norpregna-4,9-diene-3,20-dione) with the following structural formula:

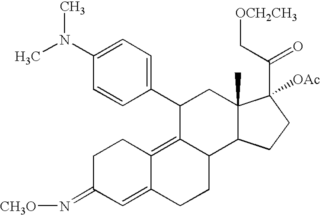

19. CDB-4223 (21-methylthio-17α-acetoxy-11β-(4 N,N-dimethylaminophenyl)-19-norpregna-4,9-diene-3,20-dione) with the following structural formula:

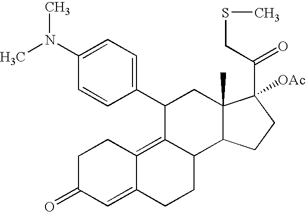

20. CDB-4119 (4-benzoin-21-acetylthio-17α-acetoxy-11β-(4 N,N-dimethylaminophenyl)-19-norpregna-4,9-diene-3,20-dione) with the following structural formula:

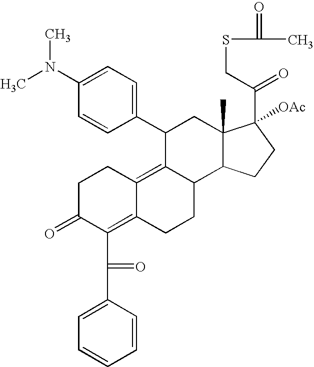

21. CDB-4239 (4-benzoin-21-methoxy-17α-acetoxy-11β-(4 N,N-dimethylaminophenyl)-19-norpregna-4,9-diene-3,20-dione) with the following structural formula:

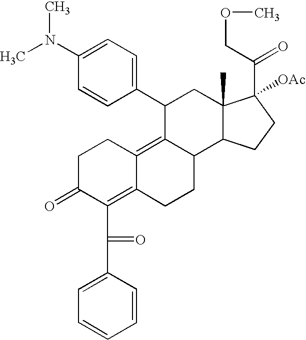

22. CDB-4306 (21-glycinate-17α-acetoxy-11β-(4 N,N-dimethylaminophenyl)-19-norpregna-4,9-diene-3,20-dione) with the following structural formula:

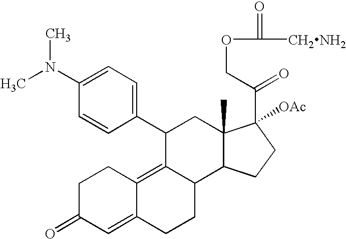

23. CDB-4352 (21-cyanothio-17α-acetoxy-11β-(4 N,N-dimethylaminophenyl)-19 norpregna-4,9-diene-3,20-dione) with the following structural formula:

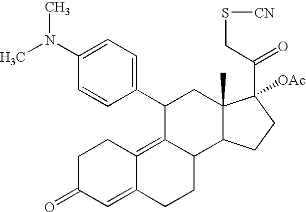

24. CDB-4362 (21-methoxyacetyl-17α-acetoxy-11β-(4 N,N-dimethylaminophenyl)-19-norpregna-4,9-diene-3,20-dione) with the following structural formula:

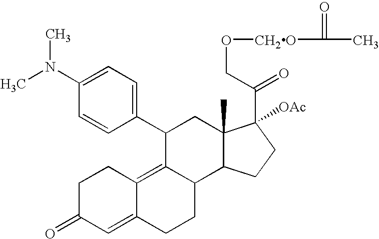

11β-monodemethylated derivatives of the 24 compounds disclosed above (i.e., those in which X is N-methylamino) are also particularly useful in practicing the instant invention. In this regard, CDB-4453 (21-methoxy-17α-acetoxy-11β-(4-N-methylaminophenyl)-19-norpregna-4,9-diene-3,20-dione), a monodemethylated derivative of CDB-4124, has been demonstrated to possess even lower anti-glucocorticoid activity than its parent. Attardi et al., 2002, Mol. Cell. Endocrin. 188:111-123, the contents of which are incorporated herein by reference.

Although compounds of the general formula above and their monodemethylated derivatives are preferred, any progesterone antagonist may be used in the practice of the present invention for its antagonist effect on the progesterone receptor. Preferably, the progesterone antagonist has one or more of the following characteristics: low antiglucocorticoid activity, minimal estrogenic and anti-estrogenic activities, and does not substantially elevate progesterone levels.

Antiprogestins which may be useful in the invention include, without limitation, asoprisnil (benzaldehyde, 4-[(11β,17β)-17-methoxy-17-(methoxymethyl)-3-oxoestra-4,9-dien-11-yl]-1-(E)-oxim; J867), its metabolite J912 (4-[17β-Hydroxy-17α-(methoxymethyl)-3-oxoestra-4,9-dien-11β-yl]benzaldehyd-(1E)-oxim), and other compounds described in DE 43 32 283 and DE 43 32 284; CDB-2914 (17α-acetoxy-11β-(4-N,N-dimethylaminophenyl)-19-norpregna-4,9-dien-3,20-dione) and other compounds described in Stratton et al., 2000, Hu. Reprod. 15:1092-1099; JNJ-1250132 and other compounds described in Allan et al., 2006, Steroids 71:949-954; 5-Aryl-1,2-dihydrochromeno[3,4-f]quinolines described in Zhi et al., 1998, J. Med. Chem. 41:291-302; 1,4-dihydro-benzo[d][1,3]oxazin-2-ones described in U.S. Pat. Nos. 6,509,334, 6,566,358 and 6,713,478 to Zhang et al.; 1,3-dihydro-indol-2-ones described in U.S. Pat. No. 6,391,907 to Fensome et al.; 2,3-dihydro-1H-indoles described in U.S. Pat. No. 6,417,214 to Ulrich et al.; benzimidazolones and analogues thereof described in U.S. Pat. No. 6,380,235 to Zhang et al.; 2,1-benzisothiazoline 2,2-dioxides described in U.S. Pat. No. 6,339,098 to Collins et al.; cyclocarbamates and cyclo-amides described in U.S. Pat. Nos. 6,306,851 and 6,441,019 to Santilli et al.; cyclic urea and cyclic amide derivatives described in U.S. Pat. No. 6,369,056 to Zhang et al.; and quinazolinone and benzoxazine derivatives described in U.S. Pat. No. 6,358,948 to Zhang et al.

Other antiprogestins that may be useful in the invention include, without limitation, (6α,11β,17β)-11-(4-dimethylaminophenyl)-6-methyl-4′,5′-dihydrospiro[estra-4,9-diene-17,2′(3′H)-furan]-3-one (ORG-31710) and other compounds described in U.S. Pat. No. 4,871,724; (11β,17α)-11-(4-acetylphenyl)-17,23-epoxy-19,24-dinorchola-4,9,20-trien-3-one (ORG-33628); (7β,11β,17β)-11-(4-dimethylaminophenyl-7-methyl]-4′,5′-dihydrospiro[estra-4,9-diene-17,2′(3′H)-furan]-3-one (ORG-31806) and other compounds described in U.S. Pat. No. 4,921,845; ZK-112993 and other compounds described in Michna et al., 1992, J. Steroid Biochem. Molec. Biol. 41:339-348; ORG-31376; ORG-33245; ORG-31167; ORG-31343; RU-2992; RU-1479; RU-25056; RU-49295; RU-46556; RU-26819; LG1127; LG120753; LG120830; LG1447; LG121046; CGP-19984A; RTI-3021-012; RTI-3021-022; RTI-3021-020; RWJ-25333; ZK-136796; ZK-114043; ZK-230211; ZK-136798; ZK-98229; ZK-98734; and ZK-137316.

Still other antiprogestins that may be useful in the invention include, without limitation, mifepristone (11β-[p-(Dimethylamino)phenyl]-17β-hydroxy-17-(1-propynyl)estra-4,9-dien-3-one; RU 486) and other compounds described in U.S. Pat. Nos. 4,386,085, 4,447,424, 4,519,946 and 4,634,695; the phosphorus-containing 17β-side chain mifepristone analogues described in Jiang et al., 2006, Steroids 71:949-954; onapristone (11β-[p-(dimethylamino)phenyl]-17α-hydroxy-17-(3-hydroxypropyl)-13α-estra-4,9-dien-3-one) and other compounds described in U.S. Pat. No. 4,780,46 1; lilopristone (((Z)-11β-[(4-dimethylamino)phenyl]-17-β-hydroxy-17α-(3-hydroxy-1-propenyl)estra-4,9-dien-3-one) and other compounds described in U.S. Pat. No. 4,609,651; the 11β-substituted 19-norsteroids, such as 11β-(4-Methoxyphenyl)-17β-hydroxy-17α-ethynyl-4,9-estradien-3-one described in Belagner et al., 1981, Steroids 37:361-382; the 11β-aryl-4-estrenes such as (Z)-11β-[(4-Dimethylamino)phenyl)]-17β-hydroxy-17α-(3-hydroxy-1-propenyl)estr-4-en-3-on described in U.S. Pat. No. 5,728,689; the 11β-aryl-estrene derivatives described in U.S. Pat. Nos. 5,843,933 and 5,843,931; the 11-benzaldoxime-estra-diene derivatives such as 4-[17β-Methoxy-17α-(methoxymethyl)-3-oxoestra-4,9-dien-11β-yl]benzaldehyde-1-(E)-oxime described in U.S. Pat. No. 5,693,628; the 11-benzaldoxime-17β-methoxy-17α-methoxymethyl-estradiene derivatives such as 4-[17β-Methoxy-17α-(methoxymethyl)-3-oxoestra-4,9-dien-11β-yl]benzaldehyde-1-(E)-[O-(ethylamino)carbonyl]oxime described in U.S. Pat. No. 5,576,310; the S-substituted 11β-benzadoxime-estra-4,9-diene-carbonic acid thiolesters such as 4-[17β-Methoxy-17α-(methoxymethyl)-3-oxoestra-4,9-dien-11β-yl]benzaldehyde-1-(E)-[O-(ethylthio)carbonyl]oxime, described in WO 99/45023; the steroid esters such as (Z)-6′-(4-cyanophenyl)-9,11α-dihydro-17β-hydroxy-17α-[4-(1-oxo-3-methylbutoxy)-1-butenyl]4′H-naphtho[3′,2′, 1′;10,9,11] estr-4-en-3-one described in DE 19652408, DE 4434488, DE 4216003, DE 4216004 and WO 98/24803; the fluorinated 17α-alkyl chain steroids such as 11β-(4-acetylphenyl)-17β-hydroxy-17α-(1,1,2,2,2-pentafluoroethyl)estra-4,9-dien-3-one described in WO 98/34947; the 17-spirofuran-3′-ylidene steroids such as 11beta-(4-Acetylphenyl)-19,24-dinor-17,23-epoxy-17alpha-chola-4,9,20-trien-3-one described in U.S. Pat. No. 5,292,878; (Z)-11beta, 19-[4-(3-Pyridinyl)-o-phenylene]-17beta-hydroxy-17α-[3-hydroxy-1-propenyl]-4-androsten-3-one and other compounds described in U.S. Pat. No. 5,439,913; the 13-alkyl-11-beta-phenyl gonanes such as 11beta-[4-(1-methylethenyl)phenyl]-17α-hydroxy-17beta-(3-hydroxypropyl)-13α-estra-4,9-dien-3-one described in U.S. Pat. No. 5,446,036; the 11-arylsteroids such as 4′,5′-Dihydro-11beta-[4-(dimethylamino)phenyl]-6beta-methylspiro[estra-4,9-dien-17beta,2′(3′H)-furan]-3-one described in U.S. Pat. No. 4,921,845; the 11-beta-aryl-estradienes described in U.S. Pat. Nos. 4,829,060, 4,814,327 and 5,089,488; the 11-beta-aryl-4,9 gonadiens and 11-beta-aryl-13-alkyl-4,9-gonadiens described in U.S. Pat. Nos.: 5,739,125, 5,407,928 and 5,273,971; the 11-beta-aryl-6-alkyl (or alkenyl or alkinyl) steroids described in EP 289073; the 10-beta,11-beta-bridged steroids described in U.S. Pat. No. 5,093,507; the 11-beta-aryl-14-beta-steroids described in U.S. Pat. No. 5,244,886; the 19,11-beta-bridged steroids described in U.S. Pat. Nos. 5,095,129, 5,446,178, 5,478,956 and 5,232,915; the 1-arylsulphonyl, arylcarbonyl and 1-arylphosphonyl-3-phenyl-1,4,5,6-tetrahydropyridazines described in U.S. Pat. No. 5,684,151; the 1-arylsulphonyl, arylcarbonyl and arylthiocarbonyl pyridazino derivatives described in U.S. Pat. No. 5,753,655; the 1,2-dihydro-[1,2-g]quinoline derivatives and 1,2-dihydro-chromeno-[3,4-f]quinoline derivatives described in U.S. Pat. Nos. 5,688,808,. 5,693,646, 5,693,647, 5,696,127, 5,696,130 and 5,696,133; the oxa-steroids 6 derived from (8S, 13S, 14R)-7-oxa-estra-4,9-diene-3,17-dione 1 described in Kang et al., 2007, Bioorg. Med. Chem. Lett. 15:907-910; and the 7-oxa-steroids 4 described in Kang et al., 2007, Bioorg. Med. Chem. Lett. 17:2531-2534.

In the preferred embodiment, the progesterone antagonist is the antiprogestin/SPRM CDB-4124 (21-methoxy-17α-acetoxy-11β-(4 N,N-dimethylaminophenyl)-19-norpregna-4,9-diene-3,20-dione). Example 10 demonstrates that administration of CDB-4124 at a high dosage (50 mg/day) suppresses endometrial thickening in adult females, but does not suppress endometrial thickening at lower dosages (25 mg/day and 12.5 mg/day).

The compounds disclosed in the instant invention may act as progesterone antagonists in the uterus. The compounds of the instant invention may be suitable for prolonged usage. Where such usage is considered, the compounds preferably have only low glucocorticoid receptor binding activity and therefore, the compounds do not substantially interfere with functions of glucocorticoid receptor. Thus, the application of the compounds may have reduced side effects, such as mood swings, fatigue and weight loss, typically found when antiprogestins with a high affinity for glucocorticoid receptor are used.

In another embodiment the instant invention teaches methods that can be used for identifying compounds that possess selective progesterone receptor binding activity. These methods include receptor binding and in vivo bioassays such as anti-McGinty, anti-Clauberg, glucocorticoid, estrogenic, androgenic, anti-glucocorticoid (AG), anti-estrogen, and anti-androgen activities as well as post-coital and anti-ovulatory activities where in the leading compounds of the instant invention are used as a reference.

In another embodiment, the instant invention teaches that the potential SPRMs can be also analyzed for their effect on transcriptional activity in human cells. When SPRMs disclosed in the instant invention are used as a reference, this analysis can furnish information about (1) SPRM's interaction with receptor, (2) interaction of the activated receptor with other transcription factors, (3) activation of a transcriptional complex at a progesterone response element (PRE); and ultimately its effect on gene expression. In these experiments, plasmid expressing the hPR-B can be cotransfected with any reporter known to a person skilled in the relevant art under the PRE-dependent promoter into HeLa, HepG2 or T47D cells. The reporters may include, but are not limited to, luciferase, beta-galactosidase, green fluorescent protein, red fluorescent protein or yellow fluorescent protein. After transfection, the cells are treated with either a test compound or one of the disclosed in this application SPRMs that serves as a positive control. Following treatment, cells are assayed for reporter expression.

In another embodiment, the instant invention teaches that prospective SPRMs can be tested for their ability to oppose dexamethasone-induced cell death in human lymphocytic cell line CEM-7 and compared to effects of SPRMs disclosed in the instant specification. In these experiments, dexamethasone can be added at a concentration that results in cell death. The cells are then treated with either RU486, one of SPRMs of the instant invention or a test compound at concentrations between 10⁻⁶ and 10⁻⁸ M.

Progesterone antagonist compounds that may be used in accordance with the present invention can be synthesized using synthetic chemistry techniques known in the art such as those disclosed in U.S. Pat. No. 6,861,415. It is to be understood that certain functional groups may interfere with other reactants or reagents under the reaction conditions and therefore may need temporary protection. The use of protecting groups is described in ‘Protective Groups in Organic Synthesis’, 2^{nd} edition, T. W. Greene & P. G. M. Wutz, Wiley-Interscience (1991).

In one embodiment, compositions of the invention comprise one or more progesterone antagonists or pharmaceutically acceptable salts thereof. Depending on the process conditions the salt compound obtained may be either in neutral or salt form. Salt forms include hydrates and other solvates and also crystalline polymorphs. Both the free base and the salts of these end products may be used in accordance with the invention.

Acid addition salts may in a manner known per se be transformed into the free base using basic agents such as alkali or by ion exchange. The free base obtained may also form salts with organic or inorganic acids.

In the preparation of acid addition salts, preferably such acids are used which form suitably pharmaceutically acceptable salts. Examples of such acids are hydrochloric acid, sulfuric acid, phosphoric acid, nitric acid, aliphatic acid, alicyclic carboxylic or sulfonic acids, such as formic acid, acetic acid, propionic acid, succinic acid, glycolic acid, lactic acid, malic acid, tartaric acid, citric acid, ascorbic acid, glucuronic acid, fumaric acid, maleic acid, hydroxymaleic acid, pyruvic acid, aspartic acid, glutamic acid, p-hydroxybenzoic acid, embonic acid, ethanesulfonic acid, hydroxyethanesulfonic acid, phenylacetic acid, mandelic acid, alogenbensenesulfonic acid, toluenesulfonic acid, galactaric acid, galacturonic acid or naphthalenesulfonic acid. All crystalline form polymorphs may be used in accordance with the invention.

Base addition salts may also be used in accordance with the invention and may be prepared by contacting the free acid form with a sufficient amount of the desired base to produce the salt in the conventional manner. The free acid form may be regenerated by contacting the salt form with an acid and isolating the free acid in the conventional manner. Pharmaceutically acceptable base addition salts are formed with metals or amines, such as alkali and alkali earth metals or organic amines. Examples of metals used as cations are sodium, potassium, calcium, magnesium and the like. Examples of suitable amines are amino acids such as lysine, choline, diethanolamine, ethylenediamine, N-methylglucamine and the like

For the aforementioned purposes, the compounds of the instant invention can be administered to a patient via any conventional route where the progesterone antagonist is active. For instance, a progesterone antagonist of the instant invention can be administered orally, parenterally, sublingually, transdermally, rectally, transmucosally, topically, via inhalation, via buccal administration, or combinations thereof. Parenteral administration includes, but is not limited to, intravenous, intraarterial, intraperitoneal, subcutaneous, intramuscular, intrathecal, intraarticular, intracisternal and intraventricular. The administration form can be a tablet, capsule, pill, nasal mist, aerosol, pellet, implant (or other depot) and the like.

A therapeutically effective amount of the composition required for use in therapy may vary depending on the particular compound employed, the mode of administration, the severity of the condition being treated, the length of time that activity is desired, among other factors, and is ultimately determined by the attendant physician. However, in general, doses employed for human treatment typically are in the range of about 0.001 mg/kg to about 500 mg/kg per day, for example about 1 μg/kg to about 1 mg/kg per day or about 1 μg/kg to about 100 μg/kg per day. For most large mammals, the total daily dosage is from about 1 to 100 mg, preferably from about 2 to 80 mg. The dosage regimen may be adjusted to provide the optimal therapeutic response. The desired dose may be conveniently administered in a single dose, or as multiple doses administered at appropriate intervals, for example as two, three, four or more subdoses per day.

Illustratively, a composition of the invention may be administered to a subject to provide the subject with a progesterone antagonist in an amount of about 1 μg/kg to about 1 mg/kg body weight, for example about 1 μg/kg, about 25 μg/kg, about 50 μg/kg, about 75 μg/kg, about 100 μg/kg, about 125 μg/kg, about 150 μg/kg, about 175 μg/kg, about 200 μg/kg, about 225 μg/kg, about 250 μg/kg, about 275 μg/kg, about 300 μg/kg, about 325 μg/kg, about 350 μg/kg, about 375 μg/kg, about 400 μg/kg, about 425 μg/kg, about 450 μg/kg, about 475 μg/kg, about 500 μg/kg, about 525 μg/kg, about 550 μg/kg, about 575 μg/kg, about 600 μg/kg, about 625 μg/kg, about 650 μg/kg, about 675 μg/kg, about 700 μg/kg, about 725 μg/kg, about 750 μg/kg, about 775 μg/kg, about 800 μg/kg, about 825 μg/kg, about 850 μg/kg, about 875 μg/kg, about 900 μg/kg, about 925 μg/kg, about 950 μg/kg, about 975 μg/kg or about 1 mg/kg body weight.

The compositions of the instant invention may contain from about 25 to about 90% of the active ingredient in combination with the carrier, more usually between about 5% and 60% by weight.

Solid carriers may include starch, lactose, dicalcium phosphate, microcrystalline cellulose, sucrose and kaolin, while liquid carriers may include sterile water, polyethylene glycols, non-ionic surfactants and edible oils such as corn, peanut and sesame oils, as are appropriate to the nature of the active ingredient and the particular form of administration desired. Flavoring agents, coloring agents, preserving agents, and antioxidants, for example, vitamin E and ascorbic acid, may be included in preparations as well. Under ordinary conditions of storage and use, the preparations may contain a preservative to prevent the growth of microorganisms.

The compositions of the instant invention can be formulated into tablets in a tablet press by using techniques well-known to an artisan skilled in the relevant field. Optionally, the active ingredients according to the invention can also be pressed separately into two-layer tablets. Compositions of the instant invention can also be formulated as an oily solution.

Patients undergoing treatments with the compositions of the instant invention should be monitored routinely for their serum estrogen and glucocorticoid levels.

The following non-limiting examples are provided to aid in understanding the teachings of the instant invention.

All patents, patent applications and publications referenced herein are hereby incorporated by reference herein to the fullest extent allowed under the law.

### EXAMPLE 1

### Formulations of The Instant Invention Can Be Prepared As Tablets

To obtain tablets for practicing the instant invention, the following ingredients can be pressed together in a tablet press:

| | |
|---|---|
| 50.0 mg | of CDB-4124 |
| 140.5 mg | of lactose |
| 69.5 mg | of corn starch |
| 2.5 mg | of poly-N-vinylpyrrolidone |
| 2.0 mg | of aerosil |
| 0.5 mg | of magnesium stearate |

To obtain oily preparations for practicing the instant invention, for example the following ingredients can be mixed together and loaded into ampoules:

| | |
|---|---|
| 100.0 mg | of CDB-4124 |
| 343.4 mg | of castor oil |
| 608.6 mg | of benzyl benzoate |

### EXAMPLE 2

### Compounds of the Instant Invention May Have Only Weak Antiglucocorticoid Receptor Binding Activity

Certain antiprogestins were tested in receptor-binding assays for their ability to bind rabbit progesterone receptor (rbPR) and glucocorticoid receptor (rbGR). Briefly, cytosol containing PR or GR were prepared in TEGMD buffer (10 mM Tris, pH 7.2, 1.5 mM EDTA, 0.2 mM sodium molybdate, 10% glycerol, 1 mM DTT) from uterus or thymus, respectively, of estradiol-primed immature rabbits. For PR binding, the cytosol was incubated with 6 nM 1,2-[³H]progesterone (50.0 Ci/mmole) and competitors were added at concentrations from 2 to 100 nM. For binding to GR, the cytosol was incubated with 6 nM 6,7-[³H]-dexamethasone (40 Ci/mmol) and test compounds were added at concentrations from 20 to 100 nM. After overnight incubation at 4 C, bound and unbound [³H] steroids were separated by addition of dextran-coated charcoal and centrifugation at 2100×g for 15 min at 4 C. Supernatants containing the [3H]-steroid receptor complexes were decanted into vials containing 4 ml Optifluor (Packard Instrument Co.), vortexed, equilibrated in a liquid scintillation counter for 30 minutes and then counted for 2 minutes. The EC₅₀ (Effective Concentration) for each standard curve and each of the compound curves was determined by entering the counting data into a four parameter sigmoidal computer program (RiaSmart® Immunoassay Data Reduction Program, Packard Instrument Co., Meriden, Conn.). Relative binding affinity (RBA) for each compound was calculated using the following equation: EC₅₀ of standard/EC₅₀ of test compound×100. The standards for the PR and GR assays were unlabeled progesterone and dexamethasone, respectively. The results of these experiments are summarized in Table 1, as a ratio of the relative binding affinities of each compound for the rbPR and rbGR receptors (rbPR/rbGR). This differential reflects the relative activity of a compound in a cell or tissue that possesses the two receptors and the requisite transcriptional cofactors.

Also given in Table 1 are the relative biological activities of the same compounds in the rabbit uterus by the anti-McGinty and anti-Clauberg assays. Compound CDB-2914 (listed at the end of the Table) was used as the control or reference compound (rabbit Biological Activity=1.00) for these experiments because results of experiments using CDB-2914 have been published before (Hild-Petito et al., 1996; Passaro et al., 1997; Reel et al., 1998; Lamer et al., 2000). For the anti-McGinty test, immature female rabbits received a subcutaneous injection of 5 μg estradiol in 10% ethanol/sesame oil daily for 6 consecutive days. On day 7, animals underwent sterile abdominal surgery to ligate a 3-4 cm segment of both uterine horns. The test compound in appropriate solvent was injected intraluminally into the ligated segment of one uterine horn and vehicle alone into the other. A stimulating dose of progesterone (267 μg/day) was administered subcutaneously to each rabbit daily for the next three days to induce endometrial proliferation. All animals were sacrificed at day 10 for removal of the uterus where a segment central to the ligatures was removed and fixed in 10% neutral buffered formalin and submitted for histological processing. Five micron sections stained with hematoxylin and cosin were evaluated microscopically for the degree of endometrial glandular proliferation. The percent inhibition of endometrial proliferation for each rabbit was calculated and the mean of the group of five animals recorded. For the Anti-Clauberg test, immature female rabbits received a subcutaneous injection of 5 μg estradiol in 10% ethanol/sesame oil daily for 6 consecutive days. On day 7, animals received progesterone by subcutaneous injection (160 μg/day) and the experimental compound in appropriate vehicle orally or subcutaneously for five consecutive days. One group of rabbits received progesterone only. Twenty-four hours after the last dose, all animals were sacrificed for removal of the uterus which was cleaned of all fat and connective tissue, weighed to the nearest 0.2 mg and placed in 10% neutral buffered formalin for subsequent histological processing. Five micron sections stained with hematoxylin and eosin were evaluated microscopically for the degree of endometrial glandular proliferation. The percent inhibition of endometrial proliferation at each dose level of the test compound was derived by comparison with progesterone-stimulated animals alone. The data presented in Table 1 (rabbit Biol. Act.) reflects the average of the results obtained for each compound by the anti-McGinty and anti-Clauberg assays relative to CDB-2914.

The tested antiprogestins were ranked on the basis of the selectivity of each compound for the rabbit PR over the rabbit GR, as listed in Table 1. The antiprogestins were also ranked on the basis of the biological activity in the rabbit uterus. Data presented in Table 1 show that the affinity of leading compounds for progesterone receptor was at least 1.5 times greater than their affinity for glucocorticoid receptor.

The results of these studies also show that the two leading compounds CDB-4124 and CDB-4059 have strong antiprogestin activity in the rabbit uterus in comparison to RU 486 and CDB-2914. Both compounds lack estrogenic, androgenic, anti-estrogenic, and anti-androgenic activities. Both compounds possess minimal anti-glucocorticoid receptor activity, a feature that distinguishes them from RU 486 and CDB-2914 which are moderately active in glucocorticoid receptor binding. In these assays, CDB-4124 performed slightly better than CDB-4059

TABLE 1
RECEPTOR BINDING AND BIOLOGICAL ACTIVITIES OF SPRMS

| | | | | | rabbit |
|---|---|---|---|---|---|
| | | rabbit Biol. | | | Biol. |
| SPRM | rbPR/rbGR | Act. | SPRM | rbPR/rbGR | Act. |
| 4239 | 14.80 | 0.60 | 4416 | 1.33 | 0.77 |
| 4241 | 9.10 | 0.34 | 4417 | 1.31 | 0.70 |
| 4361 | 7.20 | 3.03 | 4111 | 1.30 | 0.36 |
| 4306 | 5.90 | 0.95 | 4125 | 1.19 | 1.55 |
| 4363 | 5.75 | 2.53 | 4223 | 1.17 | not given |
| 3875 | 5.11 | 1.40 | 4398 | 1.16 | 0.99 |
| 4362 | 4.74 | 1.25 | 4058 | 1.08 | 0.90 |
| 4352 | 4.21 | 0.57 | 4418 | 1.03 | 0.25 |
| 4176 | 3.83 | 0.20 | 4177 | 1.03 | 0.00 |
| 4243 | 2.90 | 0.00 | 4030 | 0.96 | 0.30 |
| 4119 | 2.60 | 0.10 | 4374 | 0.95 | 2.25 |
| 4324 | 2.16 | 1.10 | 4399 | 0.93 | 0.35 |
| 4247 | 2.06 | 1.70 | 4152 | 0.82 | 1.40 |
| 4205 | 1.99 | 1.00 | 4110 | 0.70 | 0.10 |
| 4059 | 1.89 | 2.90 | 4031 | 0.69 | 0.70 |
| 4400 | 1.76 | 2.29 | 4101 | 0.61 | 0.65 |
| 3247 | 1.74 | 0.10 | 4248 | 0.42 | 0.00 |
| 4167 | 1.69 | 1.50 | 4227 | 0.38 | 0.00 |
| 4124 | 1.58 | 3.60 | 4393 | 0.35 | 0.00 |
| 4226 | 1.51 | 0.54 | 4396 | 0.18 | not given |
| 4206 | 1.44 | 0.68 | 2914 | 1.07 | 1.00 |

### EXAMPLE 3

### Measuring Cortisol

Several different experimental systems support a conclusion that RU 486 increases cortisol because RU 486 has strong anti-glucocorticoid properties in humans and primates.

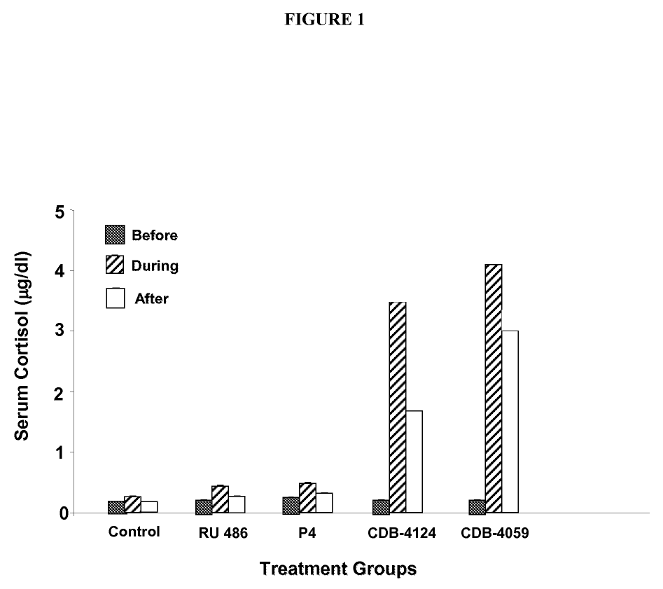

However, as shown in FIG. 1, rats treated with RU 486 at 10 mg/kg showed no significant difference in the levels of cortisol. In contrast, rats treated with either CDB-4124 or CDB-4059 at the same dose levels had significantly higher levels of serum cortisol than rats from a control group.

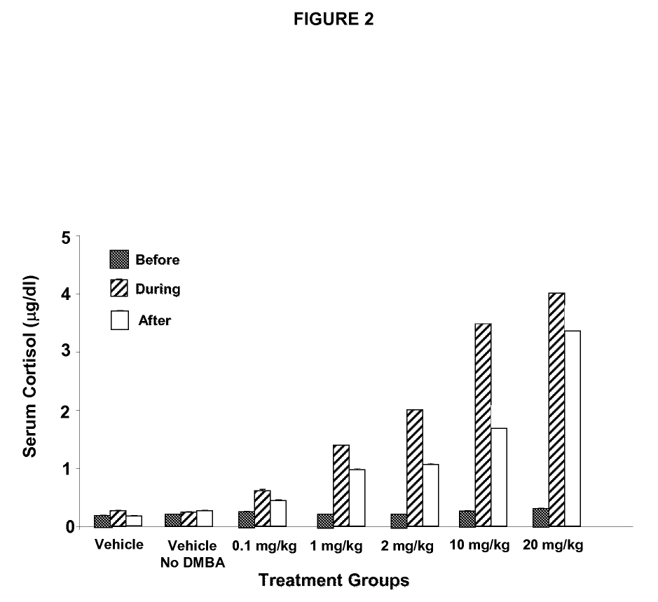

These higher levels were in the range of 3-4 ug/dl (30-40 ng/ml). The effects were dose-dependent in that increasing doses of CDB-4124 led to increased cortisol (FIG. 2).

This difference in effects of RU 486 versus CDB-4124 or CDB-4059 on cortisol levels can be explained by assuming that after 21 days of chronic dosing, a rat liver was able to metabolize RU 486 better than either of the two CDB compounds.

### EXAMPLE 4

### Measuring Corticosterone

Corticosterone is the most abundant glucocorticoid in rats. The effects of the SPRMs on cortisol shown in FIGS. 1 and 2 may be secondary to strong effects on corticosterone. To better explore this phenomenon, the levels of corticosterone were measured in groups, which showed the strongest changes in cortisol levels, such as groups treated with CDB-4124 at 20 mg/kg or 10mg/kg. For comparison, the following groups were also assayed: a group that received 20 mg/kg CDB-4124 plus 10 mg/kg progesterone, a group that received 10 mg/kg CDB-4124 plus 10 mg/kg progesterone, a group that received 10 mg/kg RU 486, a group that received 10 mg/kg of progesterone alone, a control group. The levels of corticosterone were 10-40 times higher than the levels of cortisol. However, almost no difference between groups with respect to mean corticosterone levels was observed. There were no differences among the groups before treatment (p=0.43, Kruskal-Wallis test), after 21 days of treatment (p=0.57, Kruskal-Wallis test), or after 28 days of treatment and at sacrifice (p=0.061, Kiruskal-Wallis test.

To measure effects of exogenous progesterone on serum corticosterone, the levels of corticosterone were compared in 3 paired groups that differed in whether they received exogenous progesterone (e.g., comparisons of control versus progesterone or CDB-4124 at 20 mg/kg versus CDB-4124 at 20 mg/kg plus progesterone, or CDB-4124 at 10 mg/kg versus CDB-4124 at 10 mg/kg plus progesterone). There was a statistically significant difference detected: the levels of corticosterone were lowered in animals treated with progesterone after 21 days of treatment (p=0.029, Mann-Whitney Wilcoxon test, two-tailed). This effect was not verified in sera taken at sacrifice. No differences in serum corticosterone were found between the progesterone and the CDB-4124 groups, the progesterone and the RU-486 groups, or the RU-486 group and the CDB-4124 groups.

The relationship between serum cortisol and serum corticosterone in each group was also examined. There was a strong positive linear correlation between the two for CDB-4124 at 20 mg/kg (r²=0.78), for CDB-4124 at 10 mg/kg (r²=0.82), and for RU 486 (r²=0.85). Adding progesterone to the first two CDB-4124 groups made the relationship far less strong (r²=0.34 for Group 10 and r²=0.37 for Group 11, respectively). Progesterone itself showed no such positive relationship (r²=−1.0). The control group demonstrated no relationship between the two glucocorticoids (r²=0.064). Thus, increased levels of cortisol in groups receiving CDB-4124 are correlated to levels of corticosterone, due perhaps to conversion from corticosterone that is somehow enhanced. This is consistent with an effect of CDB-4124 seen above: an effect on metabolic enzymes responsible for levels of progesterone and cortisol.

Although no strong effect of CDB-4124 on the primary glucocorticoid of the rat was found, nevertheless, for safety reasons, patients given CDB-4124 or CDB-4059 in Phase I clinical trials should be monitored for possible anti-glucocorticoid effects including a possible increase in serum cortisol, corticosterone, or ACTH.

### EXAMPLE 5

### Testing Anti-proliferative Effects of SPRMs in Uterine Cells

Any uterine cell lines can be used. Proliferation is measured in 96-well microtiter plates. 5×10³ cells are added to each well. Culture medium and drug solutions are added to wells with a Perkin Elmer Cetus PRO/PETTE. The culture medium is IMEM supplemented with 5% fetal bovine serum. Eight drug concentrations are tested, in duplicate, from 0.078 uM to 10 uM. Samples include tamoxifen alone and each of the compounds disclosed in the instant specification in combination with tamoxifen.

After a four-day incubation, the medium is replaced with fresh medium containing drug, and after a total of seven days, the cell monolayers are fixed with trichloracetic acid and stained with sulforhodamine dye. Absorbances (492 nm) of the extracted dye solutions are measured with a Titertek Multiscan plate reader. Dose response curves (percent of control absorbances vs. drug concentrations) are constructed in order to estimate IC₅₀ values defined as the drug concentrations (micromolar) which inhibited 50% proliferation. IC₅₀ values are correlative with a potency of a tested drug in inhibiting cell proliferation and therefore provide information required to identify compounds suitable for preventing hyperproliferation of the uterine cells.

### EXAMPLE 6

### CDB-4124 Lowers Luteal Phase Progesterone in Cynomolgus Monkeys

Cynomolgus monkeys (Macaca fascicularis) (n=14) were treated orally for 36 weeks with CDB-4124 or RU-486 at 1.0 mg/kg/day or with placebo (control). Another group (n=14) received Lupron® IM once per month. Urinary progesterone levels were measured for each animal for one month during the middle of the study (weeks 14-17) and for the last month of the study (weeks 33-36). The results are presented below:

| | Decrease in luteal | No decrease in luteal |
|---|---|---|
| | phase progesterone | phase progesterone |
| Controls | 1 | 13 |
| Lupron ® | 13 | 1 |
| RU 486 | 9 | 5 |
| CDB-4124 | 8 | 6 |

### EXAMPLE 7

### CDB-4124 Does Not Lower Follicular Phase Estrogen in Cynomolgus Monkeys

Urinary estrogen levels were measured for each animal of Example 6 for one month during the middle of the study (weeks 14-17) and for the last month of the study (weeks 33-36). The follicular phase results are based on 35 baseline ovulating cycles. The results are presented below:

| | | Mean | Sd | Lower? |
|---|---|---|---|---|
| Follicular Phase | | 68.3 | 19.6 | |
| Controls | Week 18 | 81.5 | 27.4 | No |
| | Week 36 | 86.3 | 23.8 | No |
| Lupron ® | Week 18 | 49.9 | 19.3 | Yes |
| | Week 36 | 41.7 | 13.4 | Yes |
| RU 486 | Week 18 | 67.4 | 27.1 | No |
| | Week 36 | 64.8 | 30.0 | No |
| CDB-4124 | Week 18 | 63.8 | 24.6 | No |
| | Week 36 | 67.3 | 22.9 | No |

### EXAMPLE 8

### CDB-4124 and Lupron® but not RU 486 Suppress Proliferation in Cynomolgus Monkey Endometrial Epithelia

At week 36, three animals from each group of Example 6 were injected within 24 hours of sacrifice with the thymidine analog bromodeoxyuridine (BrdU), a marker of proliferating cells and their progeny, to assess tissue proliferation. Full thickness uterine sections were stained and examined microscopically for evidence of proliferation in terms of the % cells positive for incorporation of BrdU:

| | Uterus epithelium | Uterus stroma | Breast |
|---|---|---|---|
| TXT | Brdu-% | Brdu-% | Brdu-% |
| Control | 10.0 ± 2.5 | 2.6 ± 0.6 | 2.4 ± 1.1 |
| Lupron ® | 3.1 ± 0.8 | 2.2 ± 1.0 | 0.3 ± 0.1 |
| RU 486 | 12.6 ± 1.8 | 3.1 ± 1.0 | 0.9 ± 0.3 |
| CDB-4124 | 2.1 ± 2.2 | 1.1 ± 0.25 | 1.9 ± 0.7 |

### EXAMPLE 9

### CDB-4124 and RU 486 but not Lupron® Enhance Apoptosis in Cynomolgus Monkey Endometrial Epithelium

Apoptosis was assessed in tissue from the same animals on slides by the terminal deoxynucleotidyl transferase mediated dUTP-biotin nick end labeling (TUNEL) technique. The percent apoptotic cells is presented below:

| | Uterus epithelium | Uterus stroma | Breast |
|---|---|---|---|
| TXT | Apo % | Apo % | Apo % |
| Control | 0.2 ± 0.1 | 0.7 ± 0.2 | 0.5 ± 0.3 |
| Lupron | 0.2 ± 0.1 | 0.2 ± 0.1 | 1.4 ± 0.7 |
| RU 486 | 0.5 ± 0.1 | 0.5 ± 0.1 | 1.2 ± 0.6 |
| CDB-4124 | 0.5 ± 0.2 | 0.5 ± 0.1 | 2.6 ± 0.9 |

### EXAMPLE 10

### CDB-4124 Suppresses Thickening in Human Endometrial Epithelia in a Dose-dependent Manner

Thirty-nine pre-menopausal adult women diagnosed with endometriosis were the subject of a six month study of Proellex™ (CDB-4124) in the treatment of endometriosis. The study included three dose levels of CDB-4124 as well as a positive control arm. The positive control was Lucrin®, a GnRH agonist, commonly used for the treatment of endometriosis (also known as Lupron®). CDB-4124 was administered in a double blinded fashion as a daily oral capsule at dosages of 12.5mg/day (n=2), 25mg/day (n=3) and 50mg/day (n=3). Another group (n=4) were injected with a slow release formulation of Lucrin® once per month as a positive control.

All doses of CDB-4124, as well as the Lucrin® dose, on average reduced distress related to pain over the course of the six month exposure to the drug, with the 50 mg CDB-4124 dose reducing both the duration and intensity of pain more effectively than the 12.5 mg or 25 mg doses and is significantly better (p=0.0012) than Lucrin® in reducing the number of days of pain over the course of the study. Pain reduction also occurred more rapidly than with the active control, Lucrin®. The response of pain to treatment in this study was analyzed in two ways. Patients in the study maintained daily pain diaries to record the severity and frequency of pain. In addition, at each office visit, patients filled out endometriosis symptom surveys that included a questionnaire that evaluated intensity of pain on a bad day on a scale of 0-10 with 10 being the greatest intensity. Daily pain diaries indicated that on average, women on Lucrin® experienced 19.4 days of pain over the first three months. Women on 50 mg of CDB-4124 exhibited less than 1 day of pain over the same period. Women on 25 mg and 12.5 mg of CDB-4124 exhibited more days of pain than that recorded by women receiving the highest dose of CDB-4124 or Lucrin®) There appeared to be a dose dependent effect on pain reduction. Over the 180 day treatment period, pain diaries indicated that women on the 50mg CDB-4124 dose had 170 or 96% pain free days (standard deviation=8.86 days). This decrease in duration of pain was statistically better (p=0.0012) than the 117.8 (74%; standard deviation 51.4 days) pain free days achieved with Lucrin n. The 50 mg dose of CDB-4124 was also statistically superior to both the 25 mg and the 12.5 mg doses with regard to pain free days. Patients on CDB-4124 12.5 mg and 25 mg doses had 115.9 (66%; standard deviation 69.2 days) and 133.6 (75%; standard deviation 27.4 days) pain free days, respectively. These results clearly support a dose response for CDB-4124. The 25 mg and 12.5 mg doses of CDB-4124 were not statistically different from Lucrin®. At the end of the first month of therapy there was a statistically significant reduction in days of pain in the 50 mg Proellex group (p=0.03 1) compared with baseline, but not in the three other treatment groups. The intensity of pain was asked by the question: “On a scale of 1-10, with 0 being no pain and 10 being extreme pain, how intense was your pain on a bad day?” The mean scores for intensity of pain at baseline were 6.3 for the CDB-4124 groups and 6.1 for the Lucrin® group. Statistically significant relief from pain was evident by the first month in the 25 mg and 50 mg Proellex groups. At month three all four active treatment groups had satistically significant reduction in pain compared with baseline, with the following scores: 3.7 (p=0.03) for 12.5 mg CDB-4124, 3.2 (p=0.03) for 25 mg CDB-4124, 1.6 (p=0.015) for 50 mg CDB-4124 and 1.5 (p=0.016) for Lucrin®. These dose related reductions continued until month six when the values for pain intensity were 2.0 (p=0.008), 2.8 (p=0.023), 0.6 (p=0.004) and 0.7 (p=0.016), respectively. Two months after stopping treatment pain returned and was of similar intensity in all four treatment groups.

Women receiving Lucrin® in the study, on average, experienced a reduction of estrogen to post-menopausal levels (<20 pg/ml) by month three and this was maintained through month six of treatment. This outcome was associated with a statistically significant increase (p=0.023) in biomarkers of bone resorption compared with the baseline values at month three, and therefore an increased risk of bone loss. At month six as well as at the one-month follow up visit, this increase in markers of bone resporption was still present in women treated with Lucrin®. All doses of CDB-4124 maintained estrogen concentrations significantly above those seen with Lucrin® and remained in the low normal range (mean >40 pg/ml). Importantly, there were no significant changes in biomarkers of bone resorption in any of the dose arms of CDB-4124 at three and six months of treatment. Women with post-menopausal levels of estrogen have been shown to be at greater risk for bone loss and other medical conditions. Lucrin®, therefore, is not indicated for treatment lasting longer than six months.

Side effects of CDB-4124 were generally mild with no individual organ system being involved systematically. Although this was a small study and no definitive conclusions can be made from the safety data, there was no single signal of safety observed.

Women in the study were closely monitored for changes in the structure of the endometrium. Data from these examinations suggest an inverse dose dependent effect of CDB-4124 on endometrial thickness at the three month period. Comparisons were made to both baseline and visit one ultrasound measurements of endometrial thickness. After three months on treatment none of the women receiving the 50 mg dose of CDB-4124 (n=3) exhibited thickened endometrium and actually exhibited a trend toward reduction in endometrial thickness compared to baseline. One woman receiving the 25 mg dose of CDB-4124 (n=4) and two women receiving the 12.5 mg dose of CDB-4124 (n=4) exhibited a thickened endometrium. The five women who received Lucrin® did not have a thickening of the endometrium due to a low estrogenic state. The results are presented below:

| | Endometrium (mm) | | |
|---|---|---|---|
| | screening | 1^{st} dose | 3 months |
| Lupron | 7.9 | 7.5 | 2.75 |
| CDB-4124 (12.5 mg) | 7.5 | 8.0 | 20.33 |
| CDB-4124 (25 mg) | 8.4 | 11.7 | 19.6 |
| CDB-4124 (50 mg) | 8.0 | 10.8 | 7.7 |

In two cases where non-menstrual spotting and bleeding was observed in patients with excessive endometrial thickening in the 12.5 mg and 25 mg CDB-4124 groups, a dilation and curettage (D&C) procedure was performed to stop the bleeding. A similar event was not seen at the 50 mg dose during the treatment phase. Greater than normal bleeding occurred in two patients in the 50 mg CDB-4124 group after treatment was stopped and a D&C was performed in one and the other successfully managed conservatively.

### EXAMPLE 11

### Treatment of Dysfunctional Uterine Bleeding using Compositions Comprising CDB-4124

Women with dysfunctional uterine bleeding are administered a once-per-day oral formulation comprising 50 mg CDB-4124. The treatment suppresses endometrial thickening, thus reducing abnormal bleeding and preventing the occurrence of anemia. The administration may be continued for 1, 2, 3, 4, 5, 6, 7, 8, 9, 12, 13, 14, 15, 16, 17, 18, 19, 20, 21, 22, 23, 24 or more months as long as desirable effects are obtained.

We claim:

1. A method for the treatment of dysfunctional uterine bleeding in a patient comprising administering to said patient an effective amount of a composition comprising a compound of formula (I): 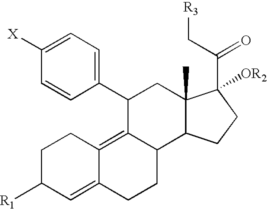
or a pharmaceutically acceptable salt, hydrate or solvate thereof, wherein:
X represents an alkyl, alkenyl, alkynyl, hydrogen, halo, monoalkylamino or dialkylamino;
R₁ represents O, NOH or NO-methyl;
R₂ represents a hydrogen or acetyl; and
R₃ represents methyloxy, formyloxy, acetoxy, acyloxy, S-alkoxy, acetyltheonyl, glycimate, vinyl ether, acethyloxymethyl, methyl carbonate, halogens, methyl, hydroxy, or ethyloxy,
wherein the amount of said composition is effective to treat dysfunctional uterine bleeding in said patient.

2. The method of claim 1, wherein said compound is CDB-4124.

3. The method of claim 2, wherein said compound is administered at a dosage from 0.5 mg/kg to 500 mg/kg.

4. The method of claim 3, wherein said compound is administered at a dosage of 50 mg per day.

5. The method of claim 1, wherein said compound is administered for a period of at least from about one to about six months.

6. The method of claim 5, wherein the endometrium of said patient is not substantially thickened during said period.

7. A method for the treatment and/or prevention of anemia in a patient with dysfunctional uterine bleeding comprising administering to said patient an effective amount of a composition comprising a compound of formula (I): 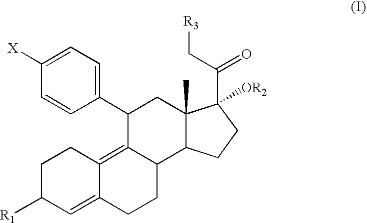
or a pharmaceutically acceptable salt, hydrate or solvate thereof, wherein:
X represents an alkyl, alkenyl, alkynyl, hydrogen, halo, monoalkylamino or dialkylamino;
R₁ represents O, NOH or NO-methyl;
R₂ represents a hydrogen or acetyl; and
R₃ represents methyloxy, formyloxy, acetoxy, acyloxy, S-alkoxy, acetyltheonyl, glycimate, vinyl ether, acethyloxymethyl, methyl carbonate, halogens, methyl, hydroxy, or ethyloxy,
wherein the amount of said composition is effective to prevent anemia in said patient.

8. The method of claim 7, wherein binding affinity of said progesterone antagonist for progesterone receptor is at least 1.5 times greater than the binding affinity of said progesterone antagonist for glucocorticoid receptor

9. The method of claim 7, wherein said compound is CDB-4124.

10. The method of claim 9, wherein said compound is administered at a dosage from 0.5 mg/kg to 500 mg/kg.

11. The method of claim 10, wherein said compound is administered at a dosage of 50 mg per day.

12. The method of claim 11, wherein said compound is administered for a period of at least from about one to about six months.

13. The method of claim 12, wherein the endometrium of said patient is not substantially thickened during said period.
